(19) United States
(12) Patent Application Publication
(10) Pub. No.: US 2024/0358358 A1
(43) Pub. Date: Oct. 31, 2024

(54) FLEXIBLE JOINT, MANUFACTURING METHOD THEREOF, AND BENDING SECTION FOR SURGICAL INSTRUMENTS

(71) Applicant: Precision Robotics (Hong Kong) Limited, Shatin, N.T. (HK)

(72) Inventor: Ning Liu, Shatin, N.T. (HK)

(21) Appl. No.: 18/687,902

(22) PCT Filed: Jul. 28, 2022
(86) PCT No.: PCT/CN2022/108608

§ 371 Date: Feb. 29, 2024

(30) Foreign Application Priority Data
Sep. 3, 2021 (HK) 22021038206.9

(51) Int. Cl. A61B 17/00; A61M 25/00; A61M 25/01

(52) U.S. Cl. CPC A61B 17/00234; A61B 2017/00314; A61B 2017/00526; A61M 25/0013; A61M 25/0138

(57) ABSTRACT

A flexible joint and a manufacturing method thereof, and a bending section for surgical instruments are provided. The flexible joint comprises a first sub-joint, two opposite sides of which are each provided with a rotation slot and a rotary joint protruding from the rotation slot; a second sub-joint provided with two protruding rotation wings corresponding to each rotation slot, and a rotation interface for the rotary joint to be embedded; the rotation wing being embedded into the rotation slot and capable of rotating along a slot wall of the rotation slot with the rotary joint as a rotation center; wherein two opposite sides of the first sub-joint are each provided with an engaging part, and the second sub-joint is provided with a corresponding engaged part; the engaging part and the engaged part being fitted with each other to prevent the first sub-joint and the second sub-joint from being separated.

### CROSS-REFERENCE TO RELATED APPLICATIONS

This Application is a National Stage Application under 35 U.S.C. § 371 of PCT Application No. PCT/CN2022/108608, filed Jul. 28, 2022 which claims priority to Hong Kong patent application Ser. No. 22021038206.9, filed Sep. 3, 2021, the entire contents of which are incorporated by reference herein.

### FIELD OF THE INVENTION

The present disclosure relates to the technical field of surgical equipment, in particular to a flexible joint made by removing useless parts of a pipe, a manufacturing method thereof, and a bending section for surgical instruments manufactured with the flexible joint.

### DESCRIPTION OF THE RELATED ART

Minimally invasive surgery (MIS), including multi-port surgery, single-port surgery, natural orifice transluminal endoscopic surgery (NOTES), etc., is becoming more and more popular. In comparison with traditional open surgery, MIS could bring patients benefits like shorter hospital stay and quicker recovery.

Flexible medical instruments are the key to the success of MIS and interventions. They can get access to interested anatomies that are difficult to reach, for example, bronchoscope can be navigated inside the complex airway networks by bending its tip to enter different branches. Besides, flexible instruments (e.g. forceps with a bendable section behind) are able to form triangulation to assist the surgeon's operation, which is crucial for single-port and endoscopic surgeries.

Various flexible joints with one or multiple bending degrees of freedom (DoFs) have been proposed to serve as the bending section of endoscopes or surgical instruments. For example, a compliant joint with multiple “segments” may be designed, and the articulation about a rotation axis may be set for each segment. These “segments” may be small-scale mechanical joints, such as ball joints, swivel joints, hinge joints, or rolling joints. Nonetheless, mechanical joints involve extremely high complexity, high material requirements under small scales, high manufacturing costs, poor reliability, and difficulty in cleaning and disinfecting.

In an existing patent with publication number CN107847280A, several technical solutions for bending section for surgical instruments not relying on traditional mechanical joints were proposed. However, these bending section for surgical instruments still involve many problems such as complex structure, high manufacturing cost, and difficulty in control and actuation.

### SUMMARY OF THE INVENTION

In order to solve or at least partially solve the above technical problems, a flexible joint made by removing useless parts of a pipe is provided in the present disclosure, comprising: a first sub-joint, two opposite sides of which are each provided with a rotation slot, and a rotary joint protruding from the rotation slot; a second sub-joint provided with two protruding rotation wings corresponding to each rotation slot, and a rotation interface for the rotary joint to be embedded which is formed between the two rotation wings; the rotation wing being embedded into the rotation slot and capable of rotating along a slot wall of the rotation slot with the rotary joint as a rotation center; wherein two opposite sides of the first sub-joint located between the two rotation slots are each provided with an engaging part, and the second sub-joint is provided with a corresponding engaged part; the engaging part and the engaged part being fitted with each other to prevent the first sub-joint and the second sub-joint from being separated.

The present disclosure further proposes a bending section for surgical instruments, which comprises the aforementioned flexible joint.

The present disclosure further proposes a method for manufacturing a flexible joint, which comprises the following steps: designing a three-dimensional graphic based on a bending part of the flexible joint; drawing a deployed plane graphic according to the deformation rules of the three-dimensional graphic deployed on an outer surface of the flexible joint; removing useless parts along a surface of the pipe, according to the plane graphic, to obtain the flexible joint.

Compared with the prior art, the present disclosure controls the degree of freedom of the flexible joint by means of the rotary joints arranged in pairs, and can realize a smooth bending action with a cooperation of the rotation wing and rotation slot, such that the bending degree of the flexible joints is controllable. A laser cutting process can be employed to remove useless parts of the pipe, making the design and manufacturing process of the flexible joint simpler, and reducing the production cost.

### DESCRIPTION OF THE DRAWINGS

To better explain embodiments of the present disclosure, relevant drawings will be briefly introduced below. It can be understood that drawings described below are only intended to illustrate some embodiments of the present disclosure, and those of ordinary skill in the art can also obtain many other technical features and connection relationships not mentioned herein, based on these drawings.

FIG. 1 is a perspective schematic view of a flexible joint in an extended state according to an embodiment of the present disclosure;

FIG. 2 is a perspective schematic view of a flexible joint in a bent state according to an embodiment of the present disclosure;

FIG. 3 is a partial schematic view of a flexible joint in a bent state according to an embodiment of the present disclosure;

FIG. 4 is a schematic view of the first step in drawing a plane graphic of a flexible joint deployed on an outer wall surface according to an embodiment of the present disclosure;

FIG. 5 is a schematic view of the second step in drawing a plane graphic of a flexible joint deployed on an outer wall surface according to an embodiment of the present disclosure;

FIG. 6 is a schematic view of the third step in drawing a plane graphic of a flexible joint deployed on an outer wall surface according to an embodiment of the present disclosure;

FIG. 7 is a schematic view showing the cutting effect in cross section of a flexible joint in an embodiment of the present disclosure when cut by a cutting device;

FIG. 8 is a schematic view showing the cutting effect in cross section of a flexible joint in an embodiment of the present disclosure when cut by another cutting device;

FIG. 9 is a schematic view showing the principle of drawing an edge of a rotation wing according to movement trajectory of the point Pi in the process of drawing a plane graphic of a flexible joint deployed on an outer wall surface in an embodiment of the present disclosure.

FIG. 10 is a schematic view of a second plane rectangular coordinate system in the process of drawing a plane graphic of a flexible joint deployed on an outer wall surface in an embodiment of the present disclosure.

### REFERENCE SIGNS

1. first sub-joint; 11. rotation slot; 111. slot wall; 12. rotary joint; 13. engaging part; 14. joint root; 2. second sub-joint; 21. rotation wing; 22. rotation interface; 23. engaged part.

### DETAILED DESCRIPTION OF THE EMBODIMENTS

The applicant has found that several technical solutions for bending section for surgical instruments not relying on traditional mechanical joints were disclosed in the prior art. However, such bending section for surgical instruments still have problems such as complex structure, high cost, and difficulty in controlling the direction and degree of bending.

Thus, the present disclosure proposes a flexible joint made by removing useless parts of a pipe and a manufacturing method thereof. Hereinafter, each embodiment of the present disclosure will be described in detail with reference to the accompanying drawings.

### First Embodiment

First embodiment of the present disclosure proposes a flexible joint made by removing useless parts of a pipe, and a method for manufacturing the flexible joint, which includes the following steps:

Step 1: designing a three-dimensional graphic based on a bending part of the flexible joint; bending manner of the bending part of the flexible joint can be clarified in a simulation software, and its performance can be estimated, according to this three-dimensional graphic.

Step 2: drawing a deployed plane graphic, according to the deformation rules of the three-dimensional graphic deployed on an outer surface of the flexible joint.

Step 3: removing useless parts along a surface of the pipe, according to the plane graphic, to obtain the flexible joint.

Once the three-dimensional graphic is deployed on an outer surface of the flexible joint, the processing problem for a structure in a three-dimensional space is converted into the processing problem for a structure in a two-dimensional plane, simplifying the problem and reducing the cost.

The pipe mentioned in the present disclosure can be made of metal or polymer materials. In order to ensure integrity of the joint, rigid materials are preferred. In this disclosure, CNC cutting technology can be employed to process the pipe, and the flexible joint can be obtained only by removing useless parts from the pipe. Since there is no need to assemble a large number of hinges or locks, production cost of the flexible joint can be significantly reduced. In addition, even when compared with other technical solutions where useless parts are removed from the pipe to obtain a flexible joint, since the pipe is simplified to a deployed plane and then part of the graphic is removed based on this plane design in the present disclosure, so compared to directly designing graphics for a curved surface of the pipe, the graphic design and the operation processes can still be significantly simplified.

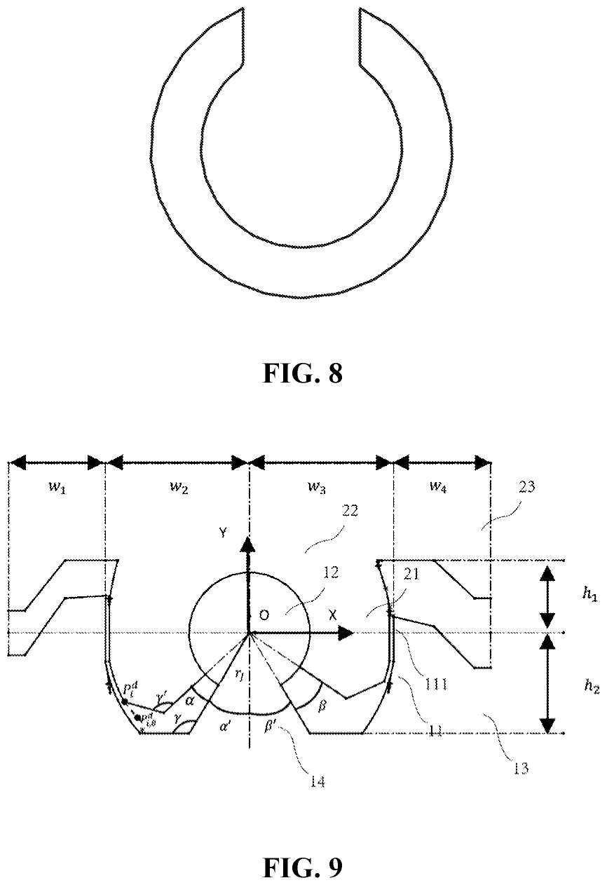

In particular, a laser cutting scheme can be employed in the present disclosure to cut out a pipe. As shown in FIG. 7, some laser cutting equipment can only cut at the center of the pipe, which may cause the cut parts to have a fan-shaped cross section, and is likely to interfere with rotational movement of the flexible joint. Therefore, a multi-axis laser cutting device is preferably employed for cutting, and the cutting is always performed perpendicular to a sectional plane of the pipe overlapping with the axis, and the cutting effect is shown in FIG. 8. In this manner, the interference to the rotational movement can be prevented to the greatest extent, and the service life of the flexible joint can be prolonged.

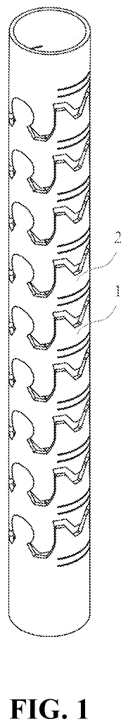

In light of this, the present disclosure further proposes a flexible joint made by removing useless parts of the pipe, as shown in FIGS. 1-3, which includes:
a first sub-joint 1, two opposite sides of which are each provided with a rotation slot 11, and a rotary joint 12 protruding from the rotation slot 12; when rotary joints 12 are arranged on two opposite sides, the direction of movement freedom of the joint is clarified, and the stability of the flexible joint is ensured;
a second sub-joint 2, which is provided with two protruding rotation wings 21 corresponding to each rotation slot 11, and a rotation interface 22 formed between the two rotation wings 21 for the rotary joint 12 to be embedded; the rotation wing 21 is embedded into the rotation slot 11 and can rotate along a slot wall 111 of the rotation slot 11 with the rotary joint 12 as a rotation center. Two opposite sides of the first sub-joint 1 located between two rotation slots 11 are provided with an engaging part 13 respectively, and the second sub-joint 2 is also provided with an engaged part 23 corresponding to the engaging part 13; the engaging part 13 and the engaged part 23 are mutually fitted to prevent separation of the first sub-joint 1 and second sub-joint 2.

In light of the above technical solutions, the present disclosure further proposes a bending section for surgical instruments which adopts the above-mentioned flexible joint. The bending section for surgical instruments of the present application is applicable to an endoscope and various flexible surgical instruments like endoscopic biopsy forceps.

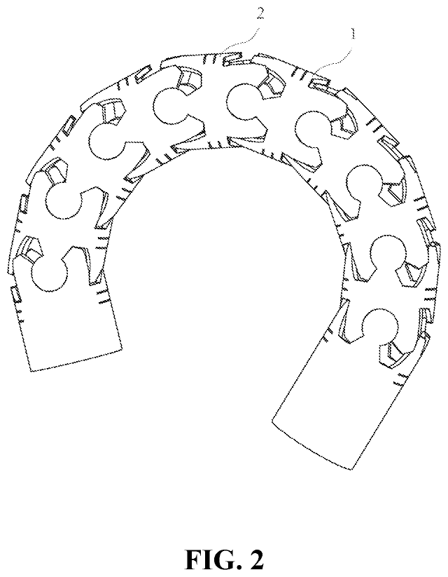

It is understood that the number of sub-joints included in the flexible joint is not limited to two. As shown in FIGS. 1-2, multiple sub-joints can be connected end to end, so as to conveniently control the length of the flexible joint and the bending amplitude of the flexible joint. In this case, upper end surface and lower end surface of each sub-joint have structures mating with each other.

By reasonably setting a gap between the rotation wing 21 and the rotation slot 11, such as making it smaller than a thickness of the pipe, two sub-joints can be effectively prevented from being separated from each other under the locking cooperation of the engaging part 13 and the engaged part 23.

Herein, as the rotation wing 21 rotates to a maximum angle, the top of the rotation wing 21 will contact the bottom of the rotation slot 11, and further movement of the rotation wing 21 is thus prevented. Optionally, the top of the rotation wing 21 and the bottom of the rotation slot 11 may be configured to contact and mate with each other, thereby improving motion stability of the rotary joint 12.

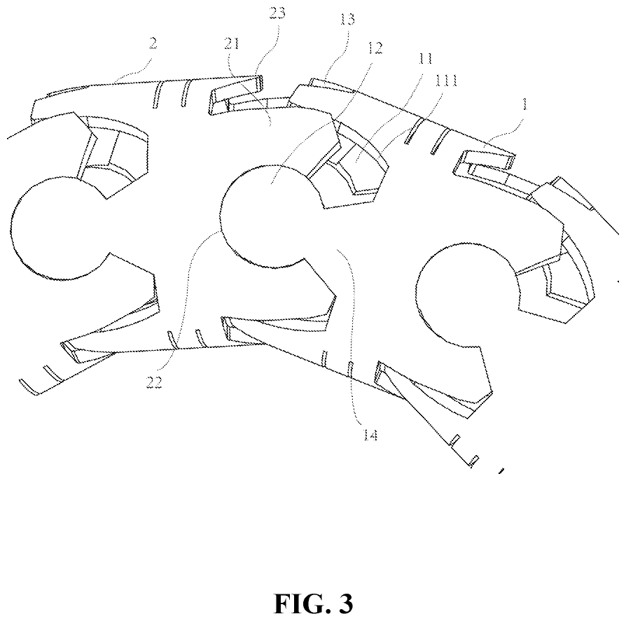

In particular, the flexible joint in the present disclosure may include a special structure as illustrated in FIG. 3. more specifically, in an embodiment of the present disclosure, the rotary joint 12 is circular, and is connected to the bottom of the rotation slot 11 via a joint root 14. In this case, an obtuse angle may be formed at a connection between the joint root 14 and the bottom of the rotation slot 11. Correspondingly, the top of rotation wing 21 may also be formed with an obtuse angle, thereby arriving at the foregoing state of contact and mating. It is to be noted that in the present disclosure, “the rotary joint 12 may be circular” does not refer to strict equivalence in terms of the geometric shape. In practice, because a surface of the pipe is curved, the rotary joint 12 cannot have a geometrically perfect circular cross-section. That is to say, the rotary joint 12 referred to in this disclosure as being circular, is in an approximate sense, and is visually close to a circular shape. By making the rotary joint 12 circular, rotation of the flexible joint can be made smoother.

Alternatively referring to FIG. 3, the engaging part 13 is formed from a groove between two protrusions formed by the slot walls 111 on both sides of the rotation slot 11, and the engaged part 23 is embedded and fitted into this groove. Obviously, under the limitation of a pair of rotary joints 12, the degree of rotation freedom of the flexible joint is constrained, and a fitting process between the engaging part 13 and the engaged part 23 will not deviate to directions other than the degree of freedom. Therefore, the engaging part 13 and the engaged part 23 can be configured to fit in a variety of ways. For example, the engaging part 13 may have a plurality of protrusions and grooves formed in a wave-shape, and the engaged part 23 engages with it correspondingly. For another example, the engaging part 13 may be formed as a protrusion, and the engaged part 23 is instead formed a groove. On the contrary, taking parts of the slot walls 111 on both sides of the rotation slot 11 as a portion of the engaging part 13 can make the structure of the flexible joint more compact.

Obviously, rotary joints 12 in the present disclosure are arranged in pairs on both sides of each sub-joint, and correspondingly, the engaging part 13 and engaged part 23 may also be provided in pairs. Two rotary joints 12 and two engaging parts 13 can respectively occupy four directions of the first sub-joint 1 in the front, rear, left and right. Correspondingly, two rotary joints 22 and two engaged parts 23 can occupy four directions of the second sub-joint 2 in the front, rear, left and right, respectively. The symmetrical arrangement scheme can also reduce design difficulty as well as the cost of the flexible joint.

Compared with the prior art, the present disclosure controls the degree of freedom of the flexible joint by means of the rotary joints 12 arranged in pairs, so that bending direction of the flexible joint is controllable. With cooperation of the rotation wing 21 and the rotation slot 11, a smooth bending action can be realized, such that the bending degree of the flexible joint can be controlled. The application of laser cutting process to remove useless parts of the pipe makes the design and manufacturing process of the flexible joint simpler and reduces the production cost.

In addition, it is understood that the flexible joint provided by the present application can be applied to a wide range of applications not only in the medical field, but also in the field of detection and repair of other industrial or domestic pipelines.

### Second Embodiment

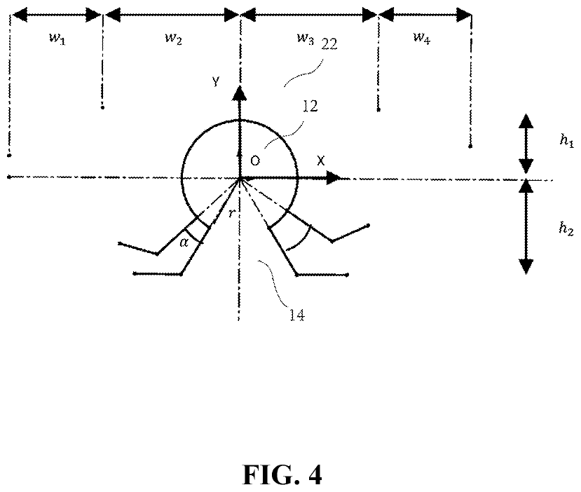

Second embodiment of the present disclosure is based on the first embodiment, and further improves the flexible joint and the manufacturing method thereof. In this embodiment, referring to FIGS. 4 to 6, the manufacturing method of the flexible joint further includes:
drawing a graphic corresponding to the rotary joint 12 of the flexible joint, which is: part of a circle; or part of an ellipse, the projection of which on the rotation plane of the flexible joint constitutes a part of a circle when the plane graphic covers an outer surface of the pipe; or a spline curve, the projection of which on the rotation plane of the flexible joint constitutes a part of a circle when the plane graphic covers an outer surface of the pipe;
drawing the remaining other graphics.

As shown in FIG. 4, drawing the remaining other graphics may include: taking a rotation center point O of the rotary joint 12 as an origin, and axial direction of the pipe as a y-axis, to establish a first plane rectangular coordinate system on the plane graphic.

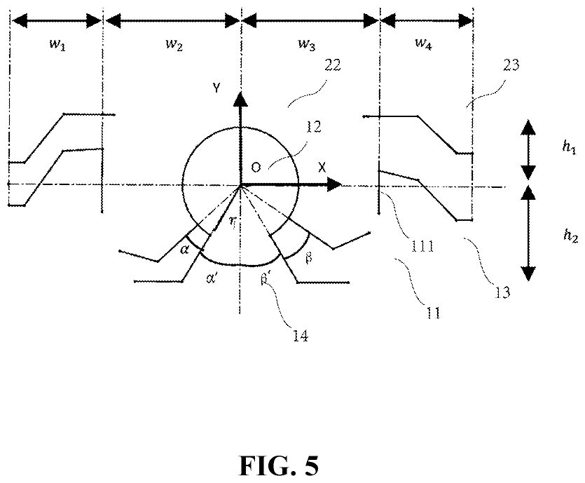

As shown in FIG. 5, two line segments parallel to the x-axis and distanced the same from the x-axis at a position about ¼ of the pipe's circumference from the O point are drawn, and a fold-line is drawn to connect these two line segments, resulting in a graphic corresponding to the engaging part 13 and the engaged part 23 of the flexible joint.

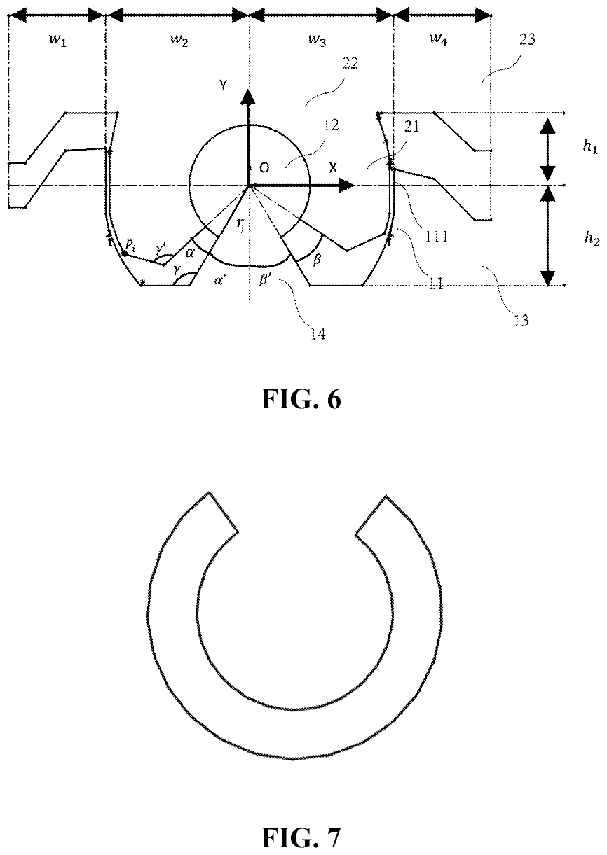

Referring to FIGS. 6 and 7, portions opposite to the rotation wing 21 and the slot wall 111 of the rotation slot 11 are drawn. Other missing lines are filled, and the plane graphic is substantially completed.

In the figure, w₁, w₂, w₃ and w₄ correspond to several areas in the plane graphic. Their width is determined by a wall width of the pipe, a cutting width W_{L} of a cutting device, material strength of the pipe, and empirical parameters, etc. Notably, if w₁ is too long and the cutting width W_{L} is too large, then the components corresponding to w₁, w₄ will not be able to well support the components corresponding to w₂ and w₃, resulting in weakening of the resistance to a lateral force, which in turn increases the probability of loss of the corresponding components.

a and B respectively represent the left and right rotation angles of the rotation wing 21, and the larger they are, the more flexible the joint is. α′ and β′ determine the size of the joint roots 14, and the larger they are, the better the mechanical strength of the joint is.

h₁ and h₂ represent an upper limit and a lower limit of the plane graphic, and their magnitudes are influenced by the values of α, β, γⱼ and l_{B}, material of the pipe, and the practical experience. The smaller the h₁ and h₂ is, the greater the number of sub-joints that can be set on the flexible joint is.

It is to be noted that this embodiment only shows a feasible process for drawing a plane graphic, and does not mean that all of the flexible joints provided in the present disclosure must be drawn with this process. Those skilled in the art can change the drawing order or other details according to actual needs.

In light of the above, as for a flexible joint as manufactured, a plane graphic of the flexible joint deployed onto an outer surface of the pipe follows the following rules. That is, the graphic corresponding to the rotary joint 12 is: part of a circle; or, part of an ellipse; or, a spline curve, the projection of which on a rotation plane of the flexible joint constitutes a part of a circle when the plane graphic covers an outer surface of the pipe.

Graphics corresponding to the rotary joint 12 and the joint root 14 are illustrated In FIG. 4. It is to be understood that once the graphic corresponding to the rotary joint 12 is removed by laser cutting, a contour corresponding to the rotary joint 12 can be obtained.

Among them, “the graphic corresponding to the rotary joint 12 is part of a circle” means that the graphic corresponding to the rotary joint 12 can be extended without changing its curvature to form a circle. When the graphic corresponding to the rotary joint 12 on the plane graphic is part of a circle, the plane graphic will inevitably be deformed as it is covered onto an outer surface of the pipe, making the shape of the rotary joint 12 itself manufactured after cutting closer to an elliptical shape. However, during rotation of an elliptical rotary joint 12, the distance from the rotary interface 22 may be close and sometimes far away, which may affect smoothness of the rotation.

Nevertheless, in the condition of drawing a graphic on a plane, if a circle is to be drawn, the amount of calculation for drawing can be greatly reduced, the convenience can be improved, and the design and production costs can be reduced. Moreover, the rotary joint 12 is located near the rotation center, and thus slight deformation has little effect. Therefore, in terms of costs, drawing the graphic corresponding to the rotary joint 12 on a plane graphic as a part of a circle is an possible option.

In addition, “the graphic corresponding to the rotary joint 12 is part of an ellipse” means that the graphic corresponding to the rotary joint 12 can be extended without changing its curvature to form an ellipse. When the graphic corresponding to the rotary joint 12 on the plane graphic is a part of an ellipse, the plane graphic will inevitably be deformed when it is covered onto an outer surface of the pipe. By adjusting a curvature of the ellipse, the shape of the rotary joint 12 itself manufactured after cutting can be made closer to a circle. Obviously, the circular rotary joint 12 owns best smoothness of rotation.

Similarly, in case of “the graphic corresponding to the rotary joint 12 is a spline curve”, the shape of the rotary joint 12 itself manufactured after cutting can also be made closer to a circle, by adjusting a curvature of the spline curve.

The technical solution employing an ellipse or a spline curve needs calculation and transformation for drawing, according to the thickness and the outer diameter of the pipe, which is relatively complicated and costly. However, it can better improve the performance of the flexible joint and extend its service life.

### Third Embodiment

Third embodiment of the present disclosure is based on the first or the second embodiment, and further improves the flexible joint and the manufacturing method thereof. In this embodiment, as shown in FIG. 5, the manufacturing method of the flexible joint, when drawing portions opposite to the rotation wing 21 and the slot wall 111 of the rotation slot 11, includes:
drawing a first straight line parallel to the y-axis;
drawing a second straight line parallel to the y-axis, as shown in FIG. 6; the distance between the two straight lines can be less than or equal to a cutting width of laser used for removing parts of the pipe; the two straight lines respectively correspond to the portions opposite to the rotation wing 21 and the slot wall 111 of the rotation slot 11; a gap between the two straight lines is a gap for the rotation wing 21 to move within the rotation slot 11. The longer these two straight lines are, the stronger lateral forces this joint can take when it is at the straight pose.
then drawing two spline curves on both sides of the straight line close to point O, still referring to FIG. 6; the distance between an intersection of extending lines of the two spline curves towards a direction of straight line close to the origin and the straight line close to the origin is less than or equal to the distance between two straight lines. These two spline curves are respectively connected to the lines corresponding to the rotary joint 12 and the engaged part 23 through a straight line or a fold-line.

The same process is taken to draw a spline curve corresponding to the straight line far from the origin, and this spline curve is in turn connected to the straight line corresponding to the bottom of the rotation slot 11. Other missing lines are filled, and the plane graphic is substantially completed.

Accordingly, in the flexible joint provided in this embodiment, one side of the rotation wing 21 facing the slot wall 111 of the rotation slot 11 has an arc-shaped edge, which also may be formed as: part of a circle; or, part of an ellipse; or, part of a spline curve.

If this edge is to be made as a part of a circle, it is necessary to deform the edge to a certain extent during processing, which may increase the difficulty of processing. Thus, this arc-shaped edge can also be formed as a part of an ellipse or a part of a spline curve.

Correspondingly, a plane graphic of the flexible joint deployed onto an outer surface of the pipe shall follow the following rules. That is, an edge of a portion of the rotation wing 21 on one side facing the slot wall 111 of the rotation slot 11 is an arc-shaped line, which is: part of a circle; or, part of an ellipse; or, a spline curve, the projection of which on a rotation plane of the flexible joint constitutes a part of a circle when the plane graphic covers an outer surface of the pipe.

Similarly in the plane graphic, when the arc-shaped edge is a part of a circle, the edge can be extended without changing its curvature to form a circle. In case the graphic corresponding to the edge of the rotation wing 21 on the plane graphic is a part of a circle, and the plane graphic is covered on an outer surface of the pipe, the formed deformation will cause the rotation wing 21 to have a shape similar to a part of an ellipse.

When the edge of the rotation wing 21 is approximately elliptical, the gap between the rotation wing 21 and the slot wall 111 of the rotation slot 11 needs to be wider, so as to avoid collision during rotation. A wider gap may decrease the endurable lateral force it can take and bring about the problem of poor service life of the components. In other words, setting the arc-shaped edge as part of a circle is a relatively degraded solution.

Similarly, in case of “the graphic corresponding to the arc-shaped edge is a spline curve”, the shape of the rotary joint 12 manufactured upon cutting can also be made closer to a circle, by adjusting a curvature of the spline curve.

Those skilled in the art can understand that a line structure of the slot wall 111 of the rotation slot 11 may be set to correspond to an edge of the rotation wing 21. This correspondence can be expressed as a correspondence in terms of shape and curvature, so that a gap between the two can be minimized and structural stability of the flexible joint can be improved.

It is understood that one side of the rotation wing 21 facing the slot wall 111 of the rotation slot 11 may present a continuous curve as a whole. Nonetheless, in the structures shown in FIGS. 5 and 6, one section of the edge of the rotation wing 21 facing the slot wall 111 of the rotation slot 11 is a straight line, and the remaining is a curved line; the straight line is located between two curved sections. In case the edge of the rotation wing 21 facing the slot wall 111 of rotation slot 11 is set as a straight line, the processing length of the curve can be reduced, the processing cost can be reduced, and the yield rate can be improved. Apparently, in the plane graphic corresponding to the rotation slot 11, sections corresponding to a straight part of the rotation wing 21 may also be a straight line. Further optionally, the distance between the two straight lines can be set as a cutting width of the equipment used for removing parts of the pipe, so that when the final cutting is performed, the distance between the two can be controlled within at least 0.5 times the cutting width. Further, while good bending performance is ensured, stability of the flexible joint can be improved, and service life of the flexible joint can be prolonged.

Further, the distance between an intersection of extending lines of the two curved lines toward the straight line and the straight line can be made smaller than the distance between the edge of the rotation wing 21 on one side facing the slot wall 111 of the rotation slot 11 and the slot wall 111 of the rotation slot 11. With this arrangement, the rotation wing 21 can be prevented from being caught by the slot wall 111 of rotation slot 11 during rotation, and rotation of the rotation wing 21 can be made smoother.

### Fourth Embodiment

Fourth embodiment of the present disclosure is based on the third embodiment, and further improves the flexible joint and the manufacturing method thereof. In this embodiment, a method for drawing a curve of the rotation wing 21 of the flexible joint and the structure of the resultant rotation wing 21 are further illustrated.

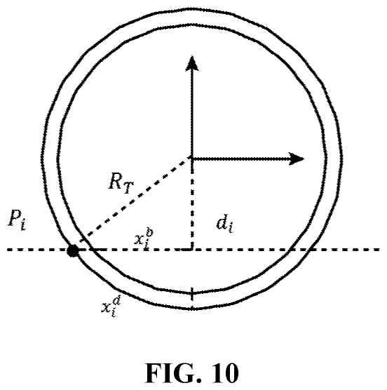

In order to draw a structure of the rotation wing 21, it is required to predict its trajectory on a drawing plane. In the deployed plane, a point corresponding to the rotation center of the rotary joint 12 is taken as an origin, and a lengthwise direction of the pipe is taken as a y-axis, to establish a first plane rectangular coordinate system; as shown in FIG. 10, in a cross section of the pipe, the center of the pipe is taken as an origin, and a direction away from the rotary joint 12 is taken as a y-axis, to establish a second plane rectangular coordinate system.

As shown in FIG. 6, for a point Pᵢ, its coordinate on the drawing plane is xᵢ^{d}yᵢ^{d}, wherein dᵢ is a distance from the tube's center to a bending plane, and therefore its coordinate on the bending plane can be obtained:

$$plane {x}_{i}^{b} = {R}_{T} \cdot sin ( {x}_{i}^{d} / {R}_{T} ) and {y}_{i}^{b} = {y}_{i}^{d} .$$

It is understood that, under basic geometric conditions, the coordinate of point Pᵢ on the bending plane after rotating about a rotation center by an angle θ is:

P_{,θ}^{b}=(x_{i,θ}^{b}, y_{i,θ}^{b}).

The definition Oᵢ^{b} is the projection of point O on a curved plane, and the current radius of rotation of the point Pᵢ in the curved plane is:

$${r}_{i}^{b} = \sqrt{{x}_{i}^{{b}^{2}} + {y}_{i}^{{b}^{2}}} .$$

The entity P_{i,θ}^{b} is deployed to return to the drawing plane, and the corresponding point is P_{i,θ}^{d}, the coordinate of which is:

$${x}_{i , θ}^{d} = {R}_{T} \cdot atan ( {x}_{i , θ}^{b} / {d}_{i} ) , {y}_{i , θ}^{d} = {y}_{i , θ}^{b} .$$

In light of the above, movement trajectory of the point P_{i,θ}^{b} on the drawing plane can be obtained. That is to say, in the plane graphic corresponding to the rotation wing 21, movement trajectory of the point P_{i,θ}^{b} that is far from the edge of the rotary joint 12 and closest to the first sub-joint 1 will satisfy:

$${x}_{i , θ}^{d} = {R}_{T} \cdot atan ( {x}_{i , θ}^{b} / {d}_{i} ) ; \\ {y}_{i , θ}^{d} = {y}_{i , θ}^{b} ;$$

where θ is an angle formed by a line connecting the point P_{i,θ}^{d} to point O and a line connecting the point Pᵢ^{d} to point O. The point Pᵢ^{d} is a position of the point P_{i,θ}^{d} itself when the rotation wing 21 has not rotated yet; x_{i,θ}^{d} is an x axis coordinate of the point P_{i,θ}^{d} in the first plane rectangular coordinate system; R_{T} is an outer diameter of the pipe; x_{i,θ}^{b} is x-axis coordinate of the point P_{i,θ}^{d} in the second plane rectangular coordinate system; dᵢ is a distance between the center of the pipe and an outer surface of the pipe; y_{i,θ}^{d} is y-axis coordinate of the point Pᵢ; and y_{i,θ}^{b} is y-axis coordinate of the point P_{i,θ}^{d} in the second plane rectangular coordinate system.

The rotation wing 21 configured in this manner can better ensure the smoothness of rotation of the rotation wing 21.

By taking advantage of the above method, the trajectories of all points of the rotation wing 21 can be obtained. In practice, however, only the trajectories of several key points need to be considered to reduce the amount of computation. The edges of the rotation slot 11 should not conflict with any of these trajectories.

### Fifth Embodiment

Fifth embodiment of the present disclosure is based on any of the first to fourth embodiments. In the fifth embodiment, a method for manufacturing a flexible joint is provided, which further includes the following steps when drawing a deployed plane graphic:
duplicating and offsetting slot wall 111 and second sub-joint 2 other than graphic arc-shaped lines corresponding to the rotary joint 12; when the lines are offset, they will move away from arc-shaped lines corresponding to the rotary joint 12 in a direction perpendicular to the axis of the pipe, and the offset amount is half of a cutting width of the cutting device used for removing parts of the pipe.

By duplicating and offsetting lines, two layers of lines for cutting can be quickly obtained. By setting the offset amount to half of a cutting width of the cutting device used for removing parts of the pipe, a gap in the final product can be controlled within a cutting error range of the cutting device, and the processing quality can be guaranteed as a result.

Finally, it shall be noted that those of ordinary skill in the art can understand that, in order to enable readers to better understand the present disclosure, many technical details are provided in the embodiments of the present disclosure. However, even without these technical details and various changes and modifications based on the foregoing embodiments, the technical solutions claimed by the claims of the present disclosure can be substantially realized. Therefore, in practical disclosures, various changes can be made to the above-mentioned embodiments in form and details, without departing from the spirit and scope of the present disclosure.

1. A flexible joint made by removing useless parts of a pipe, characterized by comprising:
a first sub-joint, two opposite sides of which are each provided with a rotation slot and a rotary joint protruding from the rotation slot;
a second sub-joint provided with two protruding rotation wings corresponding to each rotation slot, and a rotation interface for the rotary joint to be embedded which is formed between the two rotation wings; the rotation wing being embedded into the rotation slot and capable of rotating along a slot wall of the rotation slot with the rotary joint as a rotation center;
wherein two opposite sides of the first sub-joint located between the two rotation slots are each provided with an engaging part, and the second sub-joint is provided with a corresponding engaged part; the engaging part and the engaged part being fitted with each other to prevent the first sub-joint and the second sub-joint from being separated.

2. The flexible joint according to claim 1, wherein the rotary joint is circular and is connected to the bottom of the rotation slot through a joint root, and an obtuse angle is formed at the connection between the joint root and the bottom of the rotation slot; correspondingly, an obtuse angle is also formed at the top of the rotation wing; when the rotation wing rotates to a maximum angle, the top of the rotation wing and the bottom of the rotation slot coincide with each other.

3. The flexible joint according to claim 1, wherein the side of the rotation wing facing the slot wall of the rotation slot has an arc-shaped edge, wherein the edge is formed as: part of a circle; or, part of an ellipse; or, part of a spline curve.

4. The flexible joint according to claim 3, wherein one section of the edge of the rotation wing facing the slot wall of the rotation slot is a straight line, and the rest is a curve, and the straight line is located between two curves;
wherein the distance between the intersection of the extending lines of the two curves in a direction facing the straight line and the straight line is smaller than the distance between the edge of the rotation wing on the side facing the slot wall of the rotation slot and the slot wall of the rotation slot.

5. The flexible joint according to claim 1, wherein the plane graphic formed by deploying the flexible joint along the outer surface of the tube follows the following rules, that is, the edge of a part of the rotation wing on the side facing the slot wall of the rotation slot is an arc-shaped line, which is:
part of a circle; or,
part of an ellipse, wherein the projection of the ellipse on the rotation plane of the flexible joint constitutes a part of a circle; or,
a spline curve, the projection of which on the rotation plane of the flexible joint constitutes a part of a circle when the plane graphic covers the outer surface of the pipe.

6. The flexible joint according to claim 1, wherein the plane graphic formed by deploying the flexible joint along the outer surface of the pipe follows the following rules, that is, the graphic corresponding to the rotary joint is:
part of a circle; or,
part of an ellipse; or,
a spline curve, the projection of which on the rotation plane of the flexible joint constitutes a part of a circle when the plane graphic covers the outer surface of the pipe.

7. The flexible joint according to claim 6, wherein a point corresponding to the rotation center of the rotary joint on the deployed plane is taken as the origin, and the lengthwise direction of the pipe is taken as the y-axis, to establish a first plane rectangular coordinate system; in the cross section of the pipe, the center of the pipe is taken as the origin, and a direction away from the rotary joint is taken as the y-axis to establish a second plane rectangular coordinate system, then in the plane graphic corresponding to the rotation wing, the movement trajectory of the point Pie that is far from the edge of the rotary joint and closest to the first sub-joint satisfies:
$${x}_{i , θ}^{d} = {R}_{T} \cdot atan ( {x}_{i , θ}^{b} / {d}_{i} ) ; \\ {y}_{i , θ}^{d} = {y}_{i , θ}^{b} ;$$
where θ is the rotation angle of the point P_{i,θ}^{d} itself relative to the rotation wing having not rotated, x_{i,θ}^{d} is the x-axis coordinate of the point P_{i,θ}^{d} in the first plane rectangular coordinate system, R_{T} is an outer diameter of the pipe, x_{i,θ}^{b} is the x-axis coordinate of the point P_{i,θ}^{d} in the second plane rectangular coordinate system, and dᵢ is the distance from the center of the pipe to the outer surface of the pipe;
y_{i,θ}^{d} is the y-axis coordinate of the point Pᵢ, and y_{i,θ}^{b} is the y-axis coordinate of the point P_{i,θ}^{d} in the second plane rectangular coordinate system.

8. The flexible joint according to claim 6, wherein at least a part of the plane graphic corresponding to the rotation wing is a straight line, and the straight line is parallel to the axis of the pipe;
in the plane graphic corresponding to the rotation slot, the part corresponding to the straight part of the rotation wing is also a straight line.

9. The flexible joint according to claim 8, wherein the distance between the two straight lines is a cutting width of the laser used for removing parts of the pipe.

10. A bending section for surgical instruments, characterized by comprising a plurality of flexible joints according to claim 1.

11. A method for manufacturing a flexible joint, characterized by comprising the following steps:
designing a three-dimensional graphic based on a bending part of the flexible joint;
drawing a deployed plane graphic according to the deformation rules of the three-dimensional graphic deployed on an outer surface of the flexible joint;
removing useless parts along a surface of the pipe, according to the plane graphic, to obtain the flexible joint.

12. The method according to claim 11, wherein the step of drawing the deployed plane graphic further comprises:
drawing a graphic corresponding to the rotary joint of the flexible joint, which is: part of a circle; or part of an ellipse, the projection of which on the rotation plane of the flexible joint constitutes a part of a circle when the plane graphic covers the outer surface of the pipe; or a spline curve, the projection of which on the rotation plane of the flexible joint constitutes a part of a circle when the plane graphic covers the outer surface of the pipe; and
drawing the remaining other graphics.

13. The method according to claim 12, where in the step of drawing the deployed plane graphic further comprises: duplicating and offsetting slot wall 111 and second sub-joint 2 other than arc-shaped lines of the graphic corresponding to the rotary joint;
the lines being offset away from the arc-shaped lines corresponding to the rotary joint in a direction perpendicular to the axis of the pipe, and the offset amount is half of a cutting width of the cutting device used for removing parts of the pipe.
